(19) United States
(12) Patent Application Publication
(10) Pub. No.: US 2025/0099000 A1
(43) Pub. Date: Mar. 27, 2025

(54) DETERMINING LIKELIHOOD OF KIDNEY FAILURE

(71) Applicant: Roche Diagnostics Operations, Inc., Indianapolis, IN (US)

(72) Inventors: Carsten DANZER, Rombach, Aargau (CH); Martin Josef EMONS, Zürich, Zurich (CH); Martin KLAMMER, Muenchen (DE); Nicolas Seungoon SILLITOE, Allenwinden, Zug (CH); Riccardo TRIUNFO, Zug, Zug (CH)

(21) Appl. No.: 18/728,195

(22) PCT Filed: Jan. 24, 2023
(86) PCT No.: PCT/EP2023/051707

§ 371 Date: Jul. 11, 2024

(30) Foreign Application Priority Data
Jan. 28, 2022 (EP) 22154084.2

(51) Int. Cl. A61B 5/20; A61B 5/00; G16H 10/60; G16H 50/20

(52) U.S. Cl. CPC A61B 5/201; A61B 5/4842; A61B 5/7267; A61B 5/7275; G16H 10/60; G16H 50/20

(57) ABSTRACT

A computer-implemented method is provided, which determines at a prediction time tₚ, a likelihood of kidney failure of a patient within an amount of time Δt. The method comprises receiving input data, the input data comprising a recent creatinine level c_{R} or recent eGFR eGFR_{R}, and one or more of the following: (a) an initial creatinine level c₀ and either: a time t₀ at which the initial creatinine level c₀ was measured, or a time interval ΔT₀=tₚ−t₀; (b) an initial estimated glomerular filtration rate (eGFR) eGFR₀ and either: a time t₀ at which the initial eGFR was determined, or a time interval ΔT₀=tₚ−t₀; (c) for a plurality of past creatinine level measurements cᵢ measured at a respective times tᵢ, a statistical parameter derived from a linear regression of the plurality of past creatinine level measurements; and (d) for a plurality of past eGFR values eGFRᵢ determined at respective times tᵢ, a statistical parameter derived from a linear regression of the plurality of past eGFR values; and applying a machine-learning model to the input data to generate an output indicating the likelihood of kidney failure within the given amount of time Δt. Corresponding training methods and systems are also provided.

### TECHNICAL FIELD OF THE INVENTION

The present invention relates to computer-implemented methods and systems for determining a likelihood of kidney failure within an amount of time Δt. The computer-implemented method employs a machine learning model.

### BACKGROUND TO THE INVENTION

Chronic kidney disease (herein, “CKD”) is a condition in which a patient's kidneys do not work as well as they should. It is a common condition affecting vast numbers of people across the world, particularly the older population. CKD is typically caused by other conditions which result in increased strain on the kidneys, mainly high blood pressure and diabetes, along with many others.

Early stage CKD is often not readily detected; however, if detected early, a combination of lifestyle changes and medicines can be recommended, which generally result in a good prognosis for the patient.

When progressing to late stages, CKD often ultimately leads to kidney failure, thus subjecting the patient to either dialysis or kidney transplant. However, even within the population of later stage patients, CKD may progress at different speeds, and often, patients with slow progression who do not require specialist treatment but may be treated by a general practitioner, are not identified and are unnecessarily referred to a specialist, therefore putting a higher burden on the healthcare system.

CKD is often detected by creatinine measurement during routine health status tests. However, CKD often is not formally diagnosed by the physician in patients with creatinine results indicating CKD. Therefore, it is desirable to provide a way to reliably detect CKD early, especially early CKD suspected of progressing quickly, such that physicians can make appropriate recommendations as early as possible.

### SUMMARY OF THE INVENTION

Broadly speaking, the present invention provides a computer-implemented method of determining a likelihood of kidney failure within a given timescale, the computer-implemented method making use of a machine learning model and a parameter representing kidney function in a patient. The inventors have identified a set of features which, when input into an appropriate machine learning model, are able to reliably predict the likelihood of kidney failure within a predetermined time frame. In the context of the present invention, the term “kidney failure” should be understood to be synonymous with end-stage renal disorder, or the point at which dialysis or transplant are required to sustain life, i.e. when the kidneys are no longer able to provide sufficient filtration of the blood for the patient. In our data, this classification is preferably made by doctors' diagnosis codes.

More specifically, a first aspect of the invention provides a computer-implemented method of determining, at a prediction time tₚ, likelihood of kidney failure of a patient within an amount of time Δt, the computer-implemented comprising:
receiving input data, the input data comprising a recent creatinine level c_{R} or recent eGFR eGFR_{R}, and one or more of the following:
   (a) an initial creatinine level c₀ and either: a time t₀ at which the initial creatinine level c₀ was measured, or a time interval ΔT₀=tₚ−t₀;
   (b) an initial estimated glomerular filtration rate (eGFR) eGFR₀ and either: a time t₀ at which the initial eGFR was determined, or a time interval ΔT₀=tₚ−t₀;
   (c) for a plurality of past creatinine level measurements cᵢ measured at a respective times tᵢ, a statistical parameter derived from a linear regression of the plurality of past creatinine level measurements; and
   (d) for a plurality of past eGFR values eGFRᵢ determined at respective times tᵢ, a statistical parameter derived from a linear regression of the plurality of past eGFR values; and
applying a machine-learning model to the input data to generate an output indicating the likelihood of kidney failure within the given amount of time Δt.

The machine-learning model of the present invention may be a gradient-boosted decision trees algorithm or a neural network model. It should be understood that the machine-learning model which is applied to the input data is a trained machine-learning model which is configured to generate the output indicating the likelihood of kidney failure within the given amount of time Δt, based on the input data.

In the context of the present invention, the term “neural network” is used to refer to a machine-learning model (or equivalently, algorithm) which is made up of artificial neurons, or nodes. Neural networks may also be referred to as artificial neural networks, because they aim to mimic the neuronal structure of the brain. A positive weight reflects an excitatory connection, and a negative weight reflects an inhibitory connections. All inputs are modified by a weight and summed, which is referred to as a linear combination. An activation function may be used to control the amplitude of the output. Various kinds of neural network may be used in implementations of the first aspect of the invention. For example, the neural network may comprise a multi-layered perceptron (or MLP).

A MLP is a supervised learning algorithm that learns a function ƒ(⋅):R^{m}→R^{o} by training on a dataset, where m is the number of input dimensions, and o is the number of output dimensions. Given a set of features X=x₁, x₂, . . . , xₙ and a target y, it can learn a nonlinear function approximator for either classification or regression. The MLP may comprise an input layer, one or more hidden layers, and an output layer. The MLP may include two, three, four, five, six, seven, eight, nine, or ten hidden layers. Each hidden layer may include fifty or more nodes, one hundred or more nodes, two hundred or more nodes, three hundred or more nodes, four hundred or more nodes, or five hundred or more nodes. In some cases, the number of nodes in each hidden layer is a power of two. In one implementation, the MLP may comprise four hidden layers, each layer having two hundred and fifty-six nodes.

In a neural network model, each node may be associated with an activation function (or transfer function), which ultimately generates the output of the node. Various activation functions may be employed in implementations of the present invention, including: a linear activation function, a sigmoid activation function, a hyperbolic tangent activation function, a rectified linear unit (ReLU) activation function, a leaky ReLU activation function, a parameterized ReLU activation function, an exponential linear unit (ELU) activation function, a swish activation function or a softmax activation function. Another activation function which the inventors have shown to be effective is a mish activation function, defined as:

$$f ( x ) = x \cdot tanh ( ln ( 1 + {e}^{x} ) )$$

The argument of the hyperbolic tangent (i.e. ln(1+e^{x})) may be referred to as a softplus function.

More details about the manner in which the neural network model may be trained is set out later in this application.

Computer-implemented inventions according to the first aspect of the invention essentially rely on the use of data indicating the change in either a creatinine level on an eGFR over time as an input into a machine learning algorithm, which then returns a probability of kidney failure within a specified time. In some cases, input features relating, additionally or alternatively, to cystatin-c may be used in computer-implemented methods according to the first aspect of the invention (i.e. rather than or in addition to creatinine).

In some implementations of options (a) and (b), before applying the machine-learning model, the computer-implemented method may further comprise calculating slope over time using the recent creatinine level c_{R}, initial creatinine level c₀ and the time interval between the two, or the recent eGFR eGFR_{R}, the initial eGFR eGFR₀, and the time interval between the two. This slope may then also form part of the input data for the machine-learning model. In cases in which the input data comprises a statistical parameter related to a linear regression, the linear regression is preferably a linear fit which is obtained using least squares regression.

In certain cases, the “statistical parameter” referred to above may be: the slope or gradient of the linear regression (with respect to time), the intercept (i.e. on the y-axis, or the axis which represents the creatinine level or eGFR value) of the linear regression, the error (in terms of the sum of residuals) of the linear regression, the number of points considered when constructing the linear regression, and the variance of the linear regression. The input data may comprise one, all, or any subset of these statistical parameters. Alternatively, or additionally, rather than a statistical parameter being derived from a linear regression, the computer-implemented the input data may comprise another statistical parameter, for example a historical average of the creatinine level or eGFR value, a historical variance of the creatinine level or eGFR value, a historical standard deviation of the creatinine level or eGFR value, or a historical median of the creatinine level or eGFR value. Herein, “historical” refers to a statistical parameter which covers a plurality of past measurements. Other kinds of historical statistical parameters may be used too (again, alternatively or additionally).

It has been shown (as will be discussed in more detail in the “Experimental Results” section of this patent application) that the use of a machine-learning model on a minimum set of input features including a recent creatinine measurement c_{R} and one or more of (a) to (d) as set out above gives rise to reliable kidney failure likelihood determinations. In particular, the computer-implemented method of the first aspect of the invention has been shown to result in reliable likelihood determinations of kidney failure for all stages (i.e. 1 to 5) of CKD. This is advantageous over prior art methods of determining a likelihood of kidney failure in CKD patients.

The “recent” creatinine measurement should be understood to represent a creatinine measurement which was taken after any “historical” measurements. The term “recent” is not necessarily intended to specify a time frame during which the measurement should have been taken, the term is used as a label only. In some cases, the recent creatinine measurement may correspond to a most recent creatinine measurement which is available for the patient in question. The recent creatinine measurement may correspond to a measurement of the patient's creatinine level at the prediction time tₚ. In cases in which one or more statistical parameters of the linear regression are used, the linear regression may include the recent creatinine measurement. However, in some cases, the linear regression may not cover the recent creatinine measurement (i.e. the linear regression may cover all of the plurality of creatinine measurements except the most recent one, which within the meaning of this application, is the “recent creatinine measurement”). The selection of points considered when constructing a linear regression may include all points which have been measured. Alternatively, it may only include points based on measurements since a CKD diagnosis, or points going back a predetermined amount of time (e.g. 1 year, 2 years, 3 years, 4 years, or 5 years or more).

In some cases, the computer-implemented method may comprise a step of calculating a recent eGFR value from the recent creatinine measurement. Then, the input data may further comprise the recent eGFR value. Alternatively, in some cases, the input data may comprise the recent eGFR value instead of the recent creatinine measurement. Specifically, the computer-implemented invention may comprise calculating the recent eGFR value based on the recent creatinine (or alternatively, cystatin-c) measurement, the patient's age, and optionally one or more of the following: the patient's sex, the patient's race, the patient's body size (e.g. in terms of body mass index or body surface area), the patient's blood urea nitrogen measurement, and the patient's serum albumin measurement. In some cases, rather than calculating the eGFR value, the computer-implemented method may further comprise receiving an eGFR value from an external source.

Related to the previous point, in some cases, the input data may comprise both: (a) an initial creatinine level c₀ and either: a time t₀ at which the initial creatinine level c₀ was measured, or a time interval ΔT₀=tₚ−t₀; and (b) an initial estimated glomerular filtration rate (eGFR) eGFR₀ and either: a time t₀ at which the initial eGFR was determined, or a time interval ΔT₀=tₚ−t₀. In these cases, the initial eGFR value eGFR₀ may calculated from the initial creatinine (or alternatively, cystatin-c) level c₀ and additional patient data comprising age, and optionally one or more of: patient's sex, patient's race, patient's body size, the patient's blood urea nitrogen measurement, and the patient's serum albumin measurement. In these cases, the machine-learning model may be applied either to both inputs (a) and (b), or in some cases, just input (b), which indirectly includes the information from input (a).

Similarly, in some cases, the input data may comprise both: (c) for a plurality of past creatinine level measurements cᵢ measured at a respective times tᵢ, a statistical parameter derived from a linear regression of the plurality of past creatinine level measurements; and (d) for a plurality of past eGFR values eGFRᵢ determined at respective times tᵢ, a statistical parameter derived from a linear regression of the plurality of past eGFR values. In these cases, each of the plurality of past eGFR values eGFRᵢ may be calculated from the respective past creatinine level (or alternatively, cystatin-c) measurement cᵢ and additional patient data comprising age and optionally, one or more of: patient's sex, patient's race, patient's body type (e.g. in terms of body mass index and/or body surface area, where example body types include overweight, obese and very obese), the patient's blood urea nitrogen measurement, and the patient's serum albumin measurement. As before, in these cases, the machine-learning model may be applied either to both inputs (c) and (d), or in some cases, just input (d), which indirectly includes the information from input (c).

The more detailed the input data to the machine-learning model, the more reliable the prediction. With this in mind, in some cases it may be preferable for the input to contain two or more of inputs (a) to (d), three or more inputs of (a) to (d), or in some cases, all four inputs (a) to (d). Of these combinations, sets of inputs comprising (a) and (c) or (b) and (d) are preferable since they provide two different types of information, i.e. information derived from the linear regression of the creatinine level or eGFR, and information relating to an initial creatinine level or eGFR, and the time at which it was taken. It should be noted that results which are advantageous relative to known prediction techniques may be obtained with any combination of inputs (a) to (d), though.

In some cases, the input data may comprise additional features to those mentioned above. Specifically, the input data may further comprise one or more of the following: age, gender, race, albumin to creatinine ratio, serum albumin, serum cystatin-c, serum phosphate, serum bicarbonate, serum calcium, haemoglobin, glycated haemoglobin, blood urea nitrogen, number of acute kidney injury events, systolic blood pressure, diastolic blood pressure, resting heart rate, diabetes status, hypertension status, and CKD diagnosis status. Herein, when the above features refer to the name of a chemical or other species, it should be understood to mean a level or measurement of the concentration of that species in e.g. the blood, the serum, or another bodily fluid as appropriate. In computer-implemented methods according to the first aspect of the invention, the input data may include one, two, three, four, five, six, seven, eight, nine, ten, eleven, twelve, thirteen, fourteen, fifteen, sixteen, seventeen, or eighteen of the additional features set out at the beginning of this paragraph.

In a specific case, the additional features may comprise: age.

In another specific case, the additional features may comprise: albumin to creatinine ratio, haemoglobin, glycated haemoglobin, systolic blood pressure, CKD diagnosis status, gender, serum albumin, and patient's gender. The list may further include blood urea nitrogen. It should be noted that in some cases, various of these features may be used to calculate an eGFR value. In these cases, the values of these features may be taken into account both in this calculation, and the input to the machine-learning model. Alternatively, if the features are used to calculate the eGFR, then they may not (explicitly and/or directly) form input features into the machine-learning model.

In another case, the additional features may include historical creatinine or eGFR test density, which has been shown to be highly predictive. Herein, “historical creatinine or eGFR test density” refers to the frequency (e.g. in points per year) of creatinine or eGFR measurements in the patient's history, which is effectively a measure of the extent to which a patient is monitored by the healthcare system.

Computer-implemented methods according to the first aspect of the present invention have been shown to be particularly effective at predicting the onset of kidney failure over timescales of 1 to 5 years. In other words, in preferred cases, the amount of time Δt is 1 to 5 years. Alternatively put, the computer-implemented method is preferably used to determine the likelihood of kidney failure of a patient within the next 1 to 5 years. The prediction timescale may be variable, i.e. a clinician or other user may select the value of Δt for which they would like to obtain a likelihood of kidney failure. Accordingly, the input data may further comprise the value of Δt.

Traditionally, determination of the likelihood of kidney failure has only been possible (or at least, reliable) during the later stages of CKD. The computer-implemented invention of the first aspect of the present invention, however, has been shown to provide more reliable results than known methods for late stage CKD patients. Furthermore, the computer-implemented method of the first aspect of the invention is also able to provide reliable results for early stage CKD patients, or even patients who have not been diagnosed with CKD at all. Accordingly, in some cases, the patient may have been diagnosed with Stage 1 or Stage 2 CKD, or the patient may not have been diagnosed with CKD at all. Alternatively, the patient may have Stage 3 (including 3a and 3b) to 5 CKD. The stages of CKD are defined as follows:
Stage 1 (G1)—a normal eGFR above 90 ml/min, but other tests have detected signs of kidney damage
Stage 2 (G2)—a slightly reduced eGFR of 60 to 89 ml/min, with other signs of kidney damage
Stage 3a (G3a)—an eGFR of 45 to 59 ml/min
Stage 3b (G3b)—an eGFR of 30 to 44 ml/min
Stage 4 (G4)—an eGFR of 15 to 29 ml/min
Stage 5 (G5)—an eGFR below 15 ml/min, meaning the kidneys have lost almost all of their function.

The output of the invention is a likelihood of kidney failure within a time Δt. The output may comprise a probability of kidney failure within a time Δt. The probability may be presented as a value between 0 and 1, the value indicating the probability of kidney failure (where 0 indicates that there is zero likelihood, and 1 indicates that kidney failure is certain to occur within a time Δt). Similarly, the likelihood may be presented in the form of a percentage. Alternatively, or additionally, the output of the computer-implemented method may comprise a plot indicating how the likelihood varies with the value of Δt, for example in the form of a graph with the likelihood on the y-axis and the value of Δt on the x-axis. It should, of course, be stressed that such a plot would only be reliable at the time when it was generated, and that the probabilities may change as e.g. the patient's data changes over time. Alternatively, the output may comprise a score (e.g. from 0 to 10) which does not directly reflect the probability, but is correlated with the probability. This might be obtained, for example, by multiplying a probability by 10. In alternative cases, the computer-implemented method may further comprise calculating an expected time at which kidney failure is most likely to occur, based on the output of the machine-learning model.

As we have explained throughout this application, one of the main purposes of the invention is to enable a determination of whether a given patient is a “slow progressor” or a “fast progressor” as regards CKD. By making this prediction at an early stage, it is possible to better shape a patient's treatment plan. Accordingly, the computer-implemented method of the present invention may further comprise determining, based on the output of the machine-learning model, whether the patient is a fast progressor or a slow progressor. This determination may comprise comparing the output of the machine-learning model (or a value which is representative thereof, or a value calculated therefrom) with a threshold. Then, if the output (or value) is greater than the threshold (or greater than or equal to the threshold) determining that the patient is a fast progressor. And, if the output (or value) is less than the threshold (or less than or equal to the threshold), determining that the patient is a slow progressor. In some cases, where the value corresponds to e.g. an inverse or negative value representative of the likelihood, the “greater than” and “less than” may be swapped. The values of the threshold may be based on the value of Δt, and/or the stage of CKD of the patient in question.

For example, for a value of Δt of 5 years (i.e. wherein the output of the machine-learning model represents a likelihood that a patient will suffer from kidney failure within 5 years), in a Stage 3 to 5 patient, the threshold may be in the range 0.050 to 0.080, preferably 0.055 to 0.070, more preferably 0.060 to 0.065, and more preferably still about 0.020. In one embodiment, the threshold may be 0.062.

The threshold for determining rate of progressing for Stage 1 or Stage 2 patients may be slightly different than for Stage 3 to 5 patients. For example, the threshold may be in the range 0.070 to 0.100, preferably 0.075 to 0.090, and more preferably still about 0.080. In one embodiment, the threshold may be 0.081.

The values may be slightly different when Δt is 2 years. For example, in a Stage 3 to 5 patient, the threshold may be in the range 0.020 to 0.050, preferably 0.025 to 0.040, more preferably 0.030 to 0.035, and more preferably still about 0.030. In one embodiment, the threshold may be 0.032.

For Stage 1 or 2 patients, the threshold may be in the range 0.010 to 0.040, preferably 0.015 to 0.030, and more preferably still about 0.020. In one embodiment, the threshold may be 0.021.

Having a lower threshold favours false positive predictions, which is more appropriate for Stage 3 to 5 patients, where it is better to err on the side of caution and to give them more care. Having a higher threshold favours false negative predictions, which is more appropriate for Stage 1 to 2 patients, because the results of the screening should not overload the medical system with asymptomatic slowly- or non-progressing disease.

More generally, the threshold may be generated when or after the machine-learning model has been trained. Specifically, after the model has been trained on the training data (see second aspect of the invention below), the computer-implemented invention may further comprise: determining a threshold for Stage 3 to 5 patients and/or Stage 1 to 2 patients based on the training data. The threshold is preferably determined based on a specificity or sensitivity threshold. Specifically, the threshold is preferably determined such that when the machine-learning model is applied to the training data using that threshold, output meets the predetermined specificity or sensitivity thresholds. For example, the specificity threshold may be 75%, 80%, 85% or preferably 90%, or more preferably 95%. Similarly, the sensitivity threshold may be 75%, 80%, 85% or preferably 90%, or more preferably 95%. Preferably, the sensitivity threshold is used for Stage 3 to 5 patients. Similarly preferably, the specificity threshold is used for Stage 1 to 2 patients. Herein, a sensitivity of 90% may be understood to mean that the computer-implemented method of the first aspect of the invention correctly identifies 90% of fast-progressing patients as fast progressors. Herein, a specificity of 90% may be understood to mean that the computer-implemented method of the first aspect of the invention can correctly identify 90% of the slow-progressing patients as slow progressors.

The above steps relate to the use of a probability on a scale of 0 to 1. However, it will be appreciated that a similar determination can be made based on e.g. a percentage probability or a score which correlates with the probability.

The first aspect of the invention provides a computer-implemented method. Related aspects of the invention may provide, for example, data process apparatus configured to perform the computer-implemented method of the first aspect of the invention. Other related aspects include a computer program product comprising instructions which, when then program is executed by a computer, cause the computer to carry out the computer-implemented method of the first aspect of the invention. Another aspect may provide a computer-readable storage medium having the computer program product stored thereon.

The first aspect of the invention relates to the use of a machine-learning model to determine a likelihood of kidney failure within a time Δt. A second related aspect of the invention provides a computer-implemented method of generating such a model. It will be appreciated that in the computer-implemented methods provided by the first aspect of the invention, the machine-learning model may be generated using the computer-implemented method of the second aspect of the invention.

Specifically, a second aspect of the invention may provide a computer-implemented method of generating a machine-learning model configured to determine, at a prediction time tₚ, a likelihood of kidney failure of a patient within a given amount of time Δt, the computer-implemented method comprising: receiving training data, the training data comprising a plurality of data sets, representing a plurality of patients, each data set comprising input data and output data, wherein for the j^{th} data set: the input data comprises a recent creatinine level c_{j,R} (and optionally a time t_{j,R} at which it was obtained) or a recent eGFR eGFR_{j,R} (and optionally a time t_{j,R} at which it was obtained) and: (a) a historical creatinine level c_{j,H}, and the time t_{j,H} at which it was obtained; (b) a historical eGFR eGFR_{j,H}, and the time t_{j,H} at which it was obtained; (c) for a plurality of past creatinine levels measured at respective times tᵢⱼ, a statistical parameter derived from a linear regression of the plurality of past creatinine level measurements; and (d) for a plurality of past eGFR values eGFRᵢ determined at respective times tᵢ, a statistical parameter derived from a linear regression of the plurality of past eGFR values; the output data comprises an indication of an interval Δtⱼ between the time tⱼ of kidney failure, and the time of measurement of c_{j,R}; and training the machine-learning model using the training data.

Using the computer-implemented method of the second aspect of the invention enables the generation of a machine-learning model which can be used in computer-implemented methods of the first aspect of the invention. Before the step of training the machine-learning model, the computer-implemented invention may further comprise a data augmentation step. This refers to a step in which the amount of data is artificially enlarged in order to increase the volume of training data, and therefore the quality of the training of the machine-learning model. Specifically, a set of data from a single patient may be used to generate more than one piece of input data, i.e. a plurality of “snapshots” may be taken from each patient's data in order to provide additional input data items.

Alternatively put, the plurality of data sets may include one or more clusters of data sets, wherein each cluster comprises a plurality of input data items and a respective plurality of corresponding output data items, the input data items and output data items in each cluster corresponding to data obtained at different times or over different timescales for the same patient. In other words, the training data may comprise a plurality of clusters, each cluster corresponding to a respective patient, and containing input data items and corresponding output data items, each input/output pair corresponding to measurements taken at different times or over different timescales.

The data augmentation technique described above may be particularly useful to enhance the amount of data available for patients having conditions which are relatively rare. Furthermore, by using the computer-implemented methods outlined above, the machine-learning model is able to “learn” the characteristics of a patient at different time stages, e.g. at different lengths of time before kidney failure has occurred. This helps to avoid training bias. One such condition is end-stage renal disorder (ESRD), which is the final, permanent stage of CKD, where the kidney function has declined to the extent that the kidneys can no longer provide sufficient filtration of the blood for the patient. Generally, patients with ESRD are only able to survive if they receive regular dialysis or a transplant. Because this is relatively rare, it may be desirable to focus the data augmentation efforts on patients suffering from this condition. Accordingly, in some cases, the only patients for whom there is an associated cluster containing a plurality of input data items and corresponding output data items are patients who have been diagnosed with ESRD. This essentially increases the availability of ESRD training data, which is rare than non-ESRD data, and as a result, learning by the machine-learning model may be improved.

It may be necessary to take into account the “competing risk of death”, i.e. the possibility that a patient whose kidneys would have failed at a certain point dies before then for entirely different reasons. When not taking into account the competing risk of death, a non-ESRD patient is only used to make a prediction within the available data. i.e. you cannot say that a patient does not fail within 5 years if you only have 2 more years of data available.

In the spirit of the competing risk of death, a non-ESRD patient who dies in say 2 years can be used as an example of “does not fail within 5 years” because the death event can be seen as “will never reach ESRD from now on”.

So the training data changes slightly, because non-ESRD patients can be used closer to the end of their data if they died. And, the testing changes slightly as well because the algorithm can be tested on such examples as well.

The training data described above includes only data sets in which there is an indication of a time at which kidney failure actually occurred. However, in some cases, patients' kidneys will not fail. In order to improve performance of the machine-learning model, it is useful for the training data further to comprise data about patients who have not suffered from kidney failure. Accordingly, the training data may comprise a further plurality of pairs of data, wherein for the k^{th} further pair: the input data comprises a recent creatinine level c_{k,R} and one or more of: (e) a historical creatinine level c_{k,H}, and the time t_{k,H} at which it was obtained; (f) a historical eGFR eGFR_{k,H}, and the time t_{k,H} at which it was obtained; (g) for a plurality of past creatinine levels cₖᵢ measured at respective times tₖᵢ, a statistical parameter determined from a linear regression of the plurality of past creatinine level measurements; and (h) for a plurality of past eGFR values eGFRₖᵢ determined at respective times tₖᵢ, a statistical parameter derived from a linear regression of the plurality of past eGFR values; the output data comprises an indication that kidney failure has not occurred within an interval of Δtₖ since the time of measurement of c_{k,R}. By including training data in which kidney failure has not taken place, the machine-learning model is more thoroughly trained, and the resulting likelihood is likely to be more reliable as a result.

An additional aspect of the invention provides a kidney failure likelihood determination system comprising a processor which is configured to perform the computer-implemented method the first and/or second aspect of the invention. Further aspects of the invention may provide a computer program comprising instructions, which when executed by a computer (or a processor thereof), cause the computer (or processor thereof) to execute the computer-implemented method of the first and/or second aspects of the invention. In some cases, the “computer” may be a kidney failure likelihood determination system according to the previous aspect of the invention. Further aspects of the invention may provide computer-readable media comprising the computer program.

The invention includes the combination of the aspects and preferred features described except where such a combination is clearly impermissible or expressly avoided.

### BRIEF DESCRIPTION OF THE DRAWINGS

Embodiments of the present invention will now be described with reference to the accompanying drawings, in which:

FIG. 1 shows a kidney failure likelihood estimation system.

FIG. 2 is a flowchart illustrating a high-level method which may be performed by the likelihood determination module.

FIGS. 3A to 3D illustrate various types of input data for a machine-learning model which can be used to determine a likelihood of kidney failure in a patient.

FIG. 4 illustrates an example output plot which provides a prognosis over time.

FIG. 5 is a flowchart illustrating a high-level method of training a machine-learning model used to determine a likelihood of kidney failure in a patient.

FIGS. 6A to 6D illustrate various types of training input data which may be used to train a machine-learning used to determine a likelihood of kidney failure in a patient.

FIG. 7 illustrates a data augmentation process.

### DETAILED DESCRIPTION OF THE DRAWINGS

Aspects and embodiments of the present invention will now be discussed with reference to the accompanying figures. Further aspects and embodiments will be apparent to those skilled in the art. All documents mentioned in this text are incorporated herein by reference.

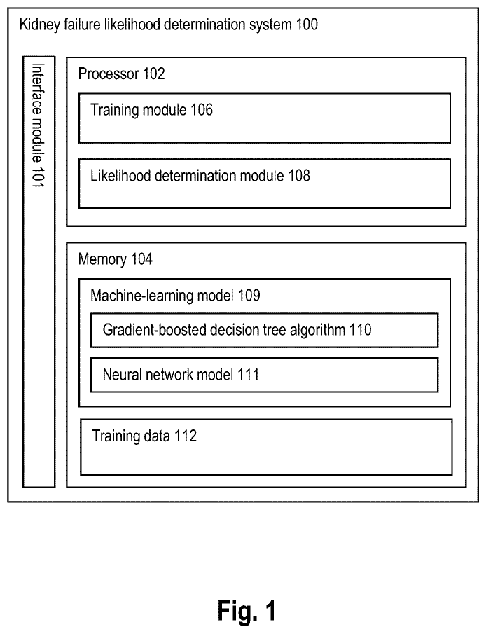

FIG. 1 shows an example of a kidney failure likelihood determination system 100 which may be used to execute computer-implemented methods according to e.g. the first and/or second aspects of the present invention. The kidney failure likelihood determination system 100 comprises an interface module 101, processor 102 and a memory 104. The processor 102 includes a training module 106 and a likelihood determination module 108. The memory 104 includes a machine-learning model 109, which may be, for example, gradient-boosted decision tree algorithm 110 and/or neural network model 111 and training data 112. It will be appreciated that other kinds of machine-learning model may also be used in the context of the present invention. FIG. 1 includes both a gradient-boosted decision tree algorithm 110 and a neural network model 111. This is illustrative only, and it should be noted that it is by no means a requirement of the invention that both of these features are included (indeed, a different machine-learning model 109) may be used.

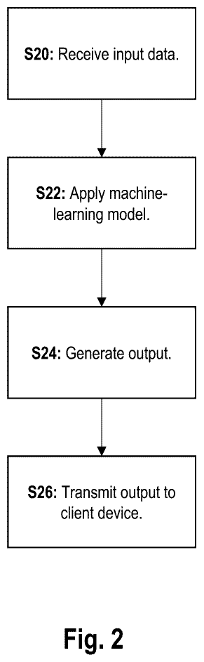

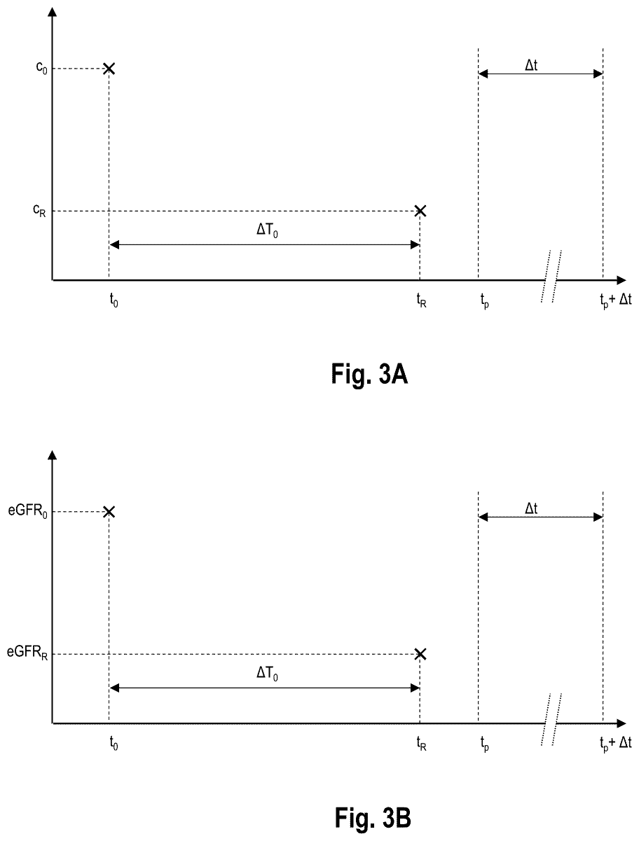

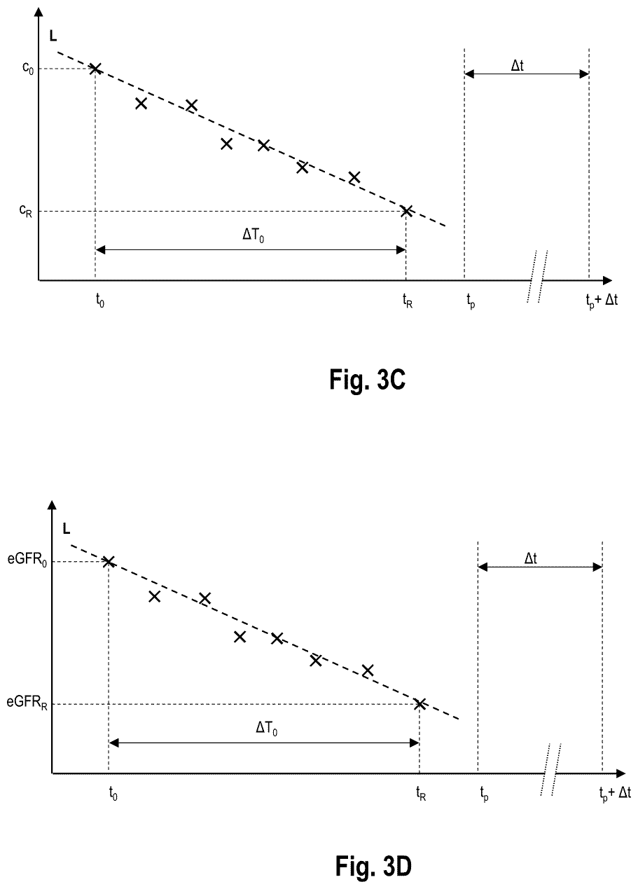

FIG. 2 is a flowchart illustrating the high-level steps which take place in a computer-implemented method according to the first aspect of the invention. In a first step S20, input data is received, for example by the interface module 101 of the kidney failure likelihood determination system 100, which acts as an interface via which information or data may be received from other, external devices (not shown). The input data which may be received in step S20 is illustrated in FIGS. 3A to 3D. In each case, the input data includes a recent creatinine measurement c_{R} and/or a recent eGFR eGFR_{R}, and additional data which may be used to estimate a trend in a patient's kidney function. More specifically:
FIG. 3A illustrates case (a), in which the input data comprises an initial creatinine level c₀ and either: a time t₀ at which the initial creatinine level c₀ was measured, or a time interval ΔT₀=tₚ−t₀.
FIG. 3B illustrates case (b), in which the input data comprises an initial estimated glomerular filtration rate (eGFR) eGFR₀ and either: a time t₀ at which the initial eGFR was determined, or a time interval ΔT₀=tₚ−t₀.
FIG. 3C illustrates case (c), in which the input data comprises, for a plurality of past creatinine level measurements cᵢ measured at respective times tᵢ, a statistical parameter derived from a linear regression of the plurality of past creatinine levels. In FIG. 3C, the line L represents the linear regression, having a negative slope s. In FIG. 3C, the line L is calculated based on a set of points which includes the recent creatinine measurement point c_{R}. However, it is envisaged that in alternative implementations, the linear regression will not include the recent creatinine measurement point c_{R}.
FIG. 3D illustrates case (d), in which the input data comprises, for a plurality of past eGFR values eGFRᵢ determined at respective times tᵢ, a statistical parameter derived from a linear regression of the plurality of past eGFR value. In FIG. 3D, the line L represents the linear regression, having a negative slope s. is calculated based on a set of points which includes the recent eGFR value eGFR_{R}. However, it is envisaged that in alternative implementations, the linear regression will not include the recent eGFR value eGFR_{R}.

The input data may further include various other measurements, or pieces of information, which are outlined earlier in this patent application.

In step S22 of FIG. 2, a machine-learning model 109 is applied to the input data, generating an output in step S24 which is indicative of the likelihood that the patient will suffer from kidney failure within a time Δt. Step S22 may be performed by the likelihood determination module 108 of the processor 102, with the machine learning model 109 being retrieved from the memory 104. With reference to the plots shown in e.g. FIGS. 3A to 3D, we illustrate what is meant by a time Δt. In computer-implemented methods of the first aspect of the invention, the likelihood determination process takes place at a time tₚ (here, p stands for “prediction”, but it should be stressed that this is a label only). In some cases, the time tₚ may be the same as the time t_{R}, at which the recent creatinine measurement or eGFR value is obtained or determined. The likelihood estimation step is then configured to determine whether kidney failure will have occurred at some time Δt after the prediction time tₚ or recent measurement time t_{R}. This time is illustrated in FIGS. 3A to 3D. It has been shown (discussed in more detail later) that computer-implemented methods according to the first aspect of the invention are effective at predicting the likelihood of kidney failure in patients for values of Δt in the range of 1 to 5 years, though larger ranges such as 1 to 10 years are also envisaged.

The output generated in step S24 may include a likelihood in the form of a probability on a scale of 0 to 1, or a percentage likelihood. In a step S26, the output may be transmitted to a client device (not shown) for display to a user such as a clinician.

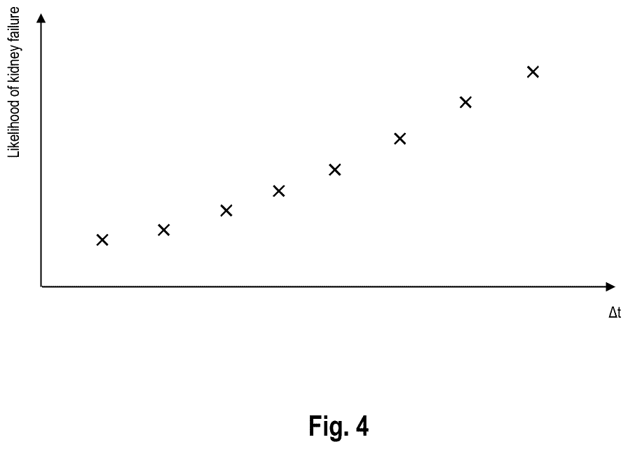

In alternative cases, steps S22 and S24 may be performed a plurality of times, for different values of Δt. In these cases, the output generated in step S24 may comprise a plot of the determined likelihood against various values of Δt, thereby illustrating the changes to a patient's prognosis over time. An example of such a plot is shown in FIG. 4.

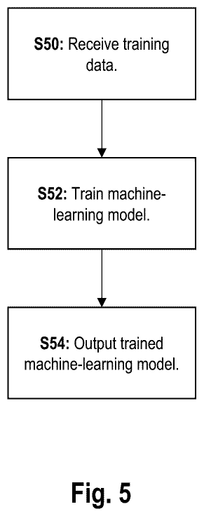

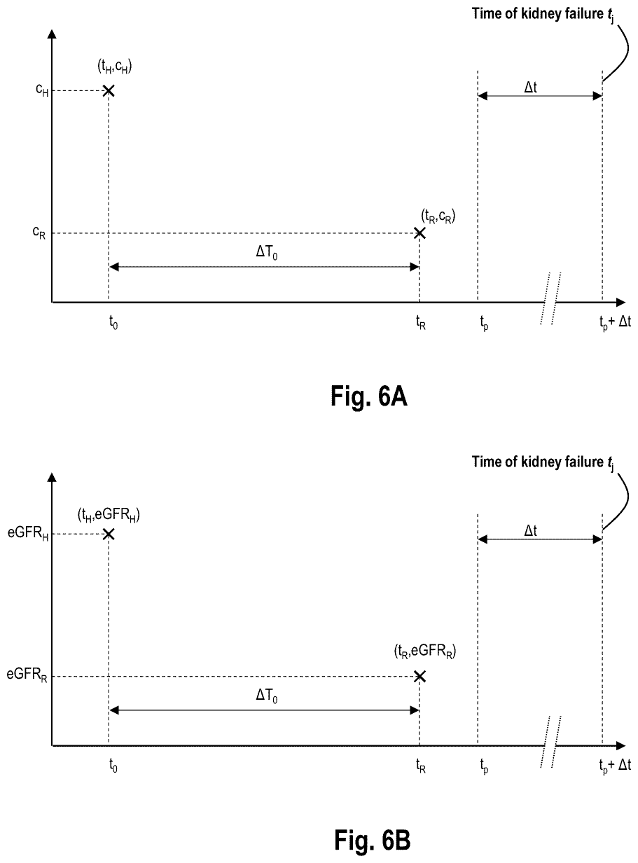

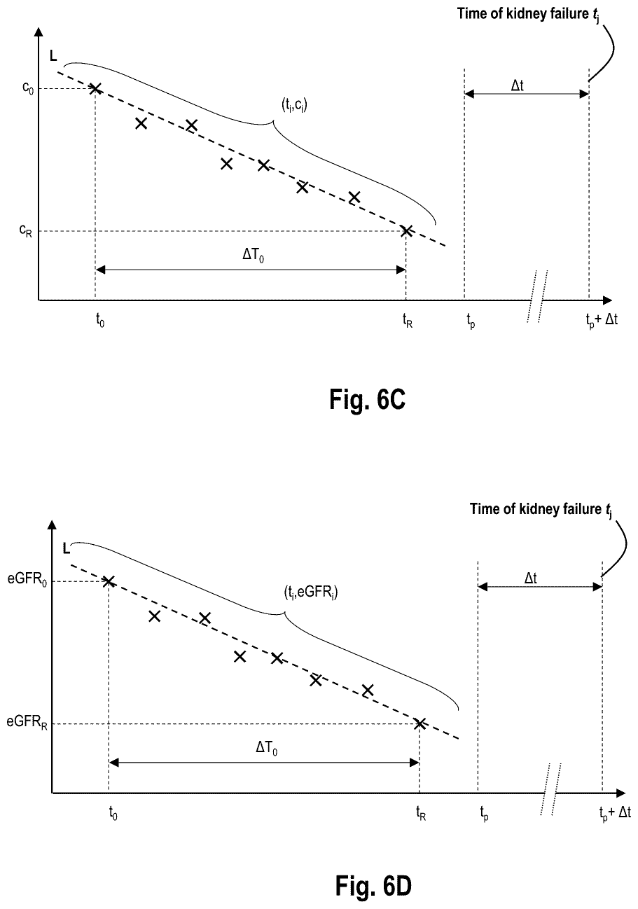

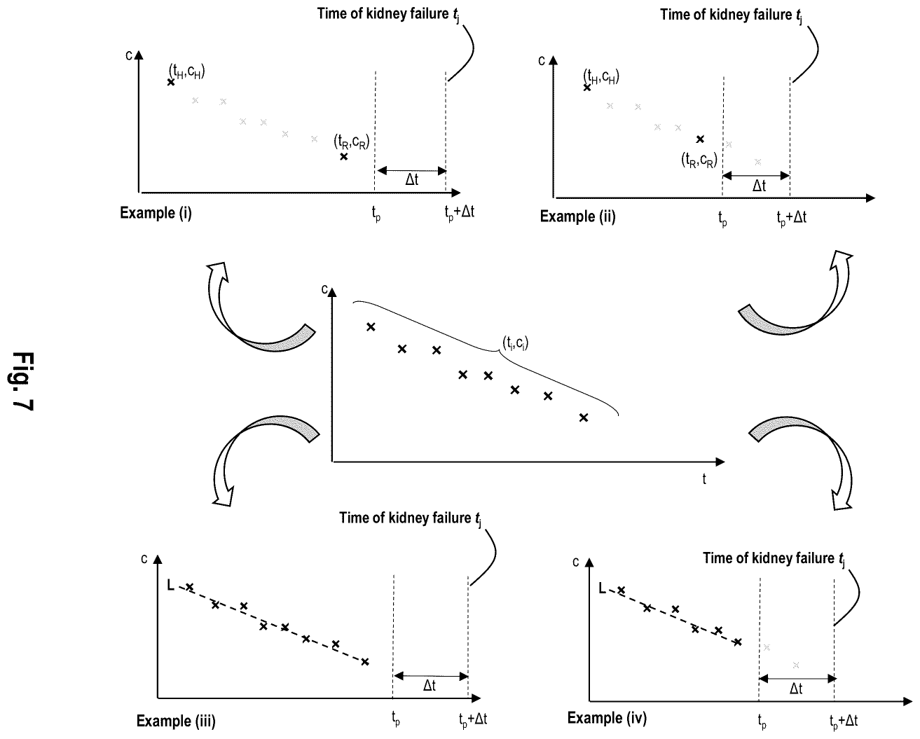

We now discuss the action of the training module 106 of the processor 102, and more specifically the types of training data 112 which may be used to train the likelihood estimation model 110. An illustrative flowchart is provided in FIG. 5. In a first step S50, training data 112 is received. This training data may be stored in memory 104 of the likelihood determination system 100. The training data may include a plurality of data sets, each data set comprising input data and output data. Broadly speaking, the input data represents values (or the like) for one or more features, which are to be input into the machine-learning model 109 in step S22 of FIG. 2. Using real patient data, it is of course not possible to assign data associated with a given patient with a likelihood of kidney failure, since this is not information which can be obtained from a patient. So, the output data may comprise an indication of either when kidney failure occurred for that patient, or in cases where no kidney failure took place, an indication of times at which kidney failure had not taken place (or relatedly, the time interval between the most recent measurement of creatinine level or eGFR value, and the time at which the data was obtained). Four types of training data are now described, with reference to FIGS. 6A to 6D. It should be stressed that training data may take other specific forms. The training data includes a plurality of j data sets, and FIGS. 6A to 6D illustrate graphically the input data which is received in an individual data set. This will be apparent to the skilled person from the drawings.
FIG. 6A illustrates a case (a) in which the input data comprises a recent creatinine level c_{R} and a time t_{R} at which it was obtained, and a historical creatinine level c_{H}, and the time t_{H} at which it was obtained. The data may take the form of at least two points in the form (t_{H}, c_{H}) and (t_{R}, c_{R}). This data could be used in order to train a machine-learning model 109 which takes input data as shown in FIG. 3A described earlier. Here, the time of the kidney event, either failure (for cases) or non-failure (for controls) tⱼ is equivalent to tₚ+Δt.
FIG. 6B illustrates a case (b) in which the input data comprises a recent eGFR eGFR_{R} and a time t_{R} at which it was obtained, and a historical eGFR eGFR_{H}, and the time t_{H} at which it was obtained. The data may take the form of at least two points in the form (t_{H}, eGFR_{H}) and (t_{R}, eGFR_{R}). This data could be used in order to train a machine-learning model 109 which takes input data as shown in FIG. 3B described earlier. Here, the time of the kidney event either failure (for cases) or non-failure (for controls) tⱼ is equivalent to tₚ+Δt.
The data in FIG. 6C illustrate a case (c) in which the input data comprises a recent creatinine level c_{R} and a time t_{R} at which it was obtained, and for a plurality of past creatinine levels measured at respective times tᵢ, a statistical parameter derived from a linear regression L of the plurality of past creatinine level measurements. The data may optionally further comprise the creatinine levels cᵢ and the associated times tᵢ. In a straightforward case, the input data from FIG. 6C could be in the form of a set of data representing one or more statistical parameters and optionally (t_{R}, c_{R}). In a simple case, this input data could be used to train a machine-learning model 109 which takes input data as shown in e.g. FIG. 3C since it provides input data in the form of a statistical parameter (e.g. the slope s or intercept of the line L though it should be noted that the data may not actually include the line L, just the raw points—the statistical parameters may be calculated during the training process). From the data shown in FIG. 6C (and indeed similar data) various additional data points can also be extracted with a view to performing data augmentation. This is discussed in more detail later in this application. Here, the time of the kidney event, either failure (for cases) or non-failure (for controls) tⱼ is equivalent to tₚ+Δt.
The data in FIG. 6D illustrate a case (d) in which the input data comprises a recent a eGFR eGFR_{R} and a time t_{R} at which it was obtained, and for a plurality of past eGFR values eGFRᵢ determined at respective times tᵢ, a statistical parameter derived from a linear regression of the plurality of past eGFR values. In a straightforward case, the input data from FIG. 6D could be in the form of a set of data representing one or more statistical parameters (e.g. the slope s or intercept of the line L though it should be noted that the data may not actually include the line L, just the raw points—the statistical parameters may be calculated during the training process) and optionally (t_{R}, eGFR_{R}). In a simple case, this input data could be used to train a machine-learning model 109 which takes input data as shown in e.g. FIG. 3D since it provides input data in the form of a statistical parameter. As with FIG. 6C, from the data shown in FIG. 6D (and indeed similar data) various additional data points can also be extracted with a view to performing data augmentation. This is discussed in more detail later in this application. Here, the time of the kidney event, either failure (for cases) or non-failure (for controls) tⱼ is equivalent to tₚ+Δt.

FIGS. 6A to 6D show examples of input data. The training data further comprises output data. This may take two forms: in the cases of patients who have suffered kidney failure, the first, an indication of a time interval between kidney failure tⱼ, and e.g. t_{R} (i.e. the time of a most recent creatinine value measurement of eGFR value determination). Alternatively the absolute time of kidney failure may be provided. In other cases, patients may not have suffered kidney failure. In this case, the output data may comprise an indication of a time tₖ at which kidney failure had not yet taken place.

Before discussing the training of the machine learning model 109 in more detail, we discuss how the training data 112 may be augmented. Performing data augmentation gives rise to a greater volume of training data 112, which itself leads to better training of the machine-learning model 109. To illustrate this principle, we refer to the plot shown in FIG. 7, which includes the same points as FIG. 6C, with the linear regression L removed, and with a time of kidney failure tⱼ shown. From a single data set such as this, it is possible to extract various sets of data which could be used to train machine-learning model 109 such as those which can be used on the data shown in FIGS. 3A and 3C:
To generate additional training data to train an algorithm which takes input data as shown in FIG. 3A, illustrated by examples (i) and (ii) in FIG. 7 (where the black data points form the input data, and the pale points are not considered): In cases in which the “real” most recent creatinine point (c_{R}, t_{R}) is used, any of the (cᵢ, tᵢ) points could be treated as the historical, or original point. The time interval to kidney failure could also be calculated straightforwardly. Furthermore, it would also be possible to use any of the (cᵢ, tᵢ) points as a most recent point (adjusting the interval to kidney failure appropriately). In this way, vastly more data may be obtained from the single data set. The plurality of data sets which may be obtained may be referred to a cluster of data sets. In example (ii), it will be noted that the earliest point is used, and an earlier point than in example (i) is used as the “most recent” creatinine measurement (and hence both tₚ and tₚ+Δt are shifted to earlier points in time, maintaining the value of Δt).
To generate additional training data to train an algorithm which takes input data as shown in FIG. 3C, illustrated by examples (iii) and (iv) in FIG. 7 (where the black data points form the input data, and the pale points are not considered): The situation here is similar, except the focus is on a statistical parameter derived from a linear regression constructed from a plurality of points. In this case, various subsets of the (cᵢ, tᵢ) points could be taken, and a statistical parameter could be calculated for each of these subsets. The interval to the time of kidney failure can then be adjusted as required. The plurality of data sets which may be obtained may be referred to a cluster of data sets. In example (iv) the most recent two creatinine measurements have been excluded from consideration (and hence both tₚ and tₚ+Δt are shifted to earlier points in time, maintaining the value of Δt).

It goes without saying that equivalent steps may be performed for machine-learning models 109 which are focused on eGFR rather than creatinine levels, and models which take input data including both eGFR and creatinine level information. As discussed earlier in the application, this can be particularly useful for generating larger volumes of training data 112 for rare types of patient, such as those suffering from ESRD.

After appropriate training data 112 has been obtained (and augmented as required), a training step takes place in step S52. At a high level, gradient-boosted decision trees algorithms 110 may be trained by iteratively reducing a loss function (e.g. cross entropy) obtained by a succession of weak learners (e.g. “stumps”, which are single-split decision trees). Detailed information about training gradient-boosted decision trees algorithms may be found in Chen & Guestrin (2016)¹, which focuses on XGBoost; Prokhorenkova et al. (2019)², which focuses on CatBoost; and Ke et al. (2017)³, which focuses on LightGBM. For the avoidance of doubt, all of these publications are incorporated herein by reference. ¹ Chen & Guestrin “XGBoost: A Scalable Tree Boosting System” (2016) arXiv:1603.02754² Prokhorenkova et al. “CatBoost: unbiased boosting with categorical features” (2019) arXiv:1706.09516³ Ke, Guolin, et al. “Lightgbm: A highly efficient gradient boosting decision tree.” Advances in neural information processing systems 30 (2017): 3146-3154.

A neural network model 111 such as a multi-layer perceptron may be trained using an Adam optimizer on a multitarget cross entropy loss.

After sufficient training in step S52, the complete (i.e. trained) machine-learning model 109 is output in step S54. This algorithm may then be used to perform computer-implemented methods according to the first aspect of the invention.

Having now described the training and operation of the machine-learning model 109, in the next section, we present evidence for the efficacy of such algorithms, specifically a gradient-boosted decision trees algorithm 110 and a neural network model 111.

### Experimental Methods & Results—Gradient-Boosted Decision Trees

### A. Training Data

To develop and train the CKD risk prediction algo, real world data (RWD) was obtained from a database. The database contains longitudinal electronic health records (EHR) as well as medical insurance claims. A relevant subset of more than 250,000 patients was used. In total, 49 lab parameters, vital signs, demographics and diagnosis codes were extracted from this database, to serve as input features for the gradient-boosted decision tree algorithms utilized in embodiments of the present invention. Chronic kidney disease (CKD) patients were identified by searching for ICD9 and ICD10 codes that are related to CKD (585 and N18, respectively) and had at least one measurement of serum creatinine in their EHR. The CKD staging (Stages 1-5) was performed based on recalculated estimated glomerular filtration rate (eGFR) values using the FAS formula (see Pottel et al. (2016)⁴). Patients with kidney failure were identified based on their medical claims data and diagnosis codes by searching for claims or diagnosis codes related to dialysis or kidney transplant and by looking for consistently low eGFR values. ⁴ Pottel, Hans et al. “An estimated glomerular filtration rate equation for the full age spectrum.” Nephrology, dialysis, transplantation: official publication of the European Dialysis and Transplant Association—European Renal Association vol. 31, 5 (2016): 798-806. doi:10.1093/ndt/gfv454

### B. Verification Data

To test the trained model with yet unseen data, patient information was obtained from a second database. In total, data for more than 650,000 relevant patients was available and the data preprocessing was performed similarly to the data base used for training.

### C. Algorithm Training

The CKD risk prediction algo employs a gradient-boosted decision tree model (in this case the CatBoost implementation of Prokhorenkova et al. (2019)⁵), which has been shown to exhibit good performance on tabulated data such as the patient data extracted from the RWD databases. The availability of longitudinal data allowed for different ways to aggregate or transform temporal feature data (e.g. single measurement value closest to the prediction time point, the variance of measurements over a certain time interval, the trend represented by the slope of a linear regression over a certain time interval, etc.), as explained with reference to FIG. 7 of the present application. This feature engineering process increased the number of available features to a total number 87. Thousands of feature combinations were systematically evaluated and a core set of particularly important features was selected. Including additional features can further improve the prediction performance, however it was shown that the magnitude of improvement becomes smaller by each additional feature added. ⁵ Prokhorenkova et al. “CatBoost: unbiased boosting with categorical features” (2019) arXiv:1706.09516

The performance of the CKD risk algo was assessed by employing 3-fold cross validation on the training data. In parallel, the reference method (the so-called “Kidney failure risk equation”, KFRE; see Tangri et al (2011)⁶) was assessed on the same data set and the performances were compared by means of the area under the receiver operating characteristics curve (AUROC or AUC). ⁶ Tangri, Navdeep et al. “A predictive model for progression of chronic kidney disease to kidney failure.” JAMA vol. 305, 15 (2011): 1553-9. doi: 10.1001/jama.2011.451

### D. Algorithm Verification

To test the performance of the CKD risk algorithm on a second independent data set and compare the results to the reference method (KFRE) in an unbiased way, the CKD risk algo was trained on the full training data and tested with the verification data. In parallel, KFRE was also applied to the verification data and the prediction results were compared by means of AUROC.

### E. Results

“Creatinine” (corresponding to the recent creatinine level) and “Creatinine_Slope” were identified as a core features set “Core Set 1”, which covers options (c) and (d) of claim 1. Additional informative features are as follows, referred to collectively below as “add'l features”.
Albumin (albumin level)
Last_ACR (recent albumin-to-creatinine ratio)
CKD Diag (CKD diagnosis status)
HbA1c (glycated haemoglobin level)
Haemoglobin (haemoglobin level)
BP Systolic (systolic blood pressure)
Patient_gender (the gender of the patient)

Results are shown in the table below, for a prediction for Δt=5 years. These results were obtained from patients with Stage 3 to 5 CKD.

| Feature set | Training data | Verification data |
|---|---|---|
| Core set 1 | CKD algo: AUC = 0.949 | CKD algo: 0.918 |
| | KFRE: AUC = 0.893 | KFRE: AUC = 0.896 |
| Core set 1 + | CKD algo: AUC = 0.951 | CKD algo: AUC = 0.92 |
| age | KFRE: AUC = 0.893 | KFRE: AUC = 0.896 |
| Core set 1 + | CKD algo: AUC = 0.963 | CKD algo: AUC = 0.936 |
| add'l features | KFRE: AUC = 0.893 | KFRE: AUC = 0.896 |

Core set 2 refers to a combination of a recent creatinine level and a historical creatinine value (and implicitly, the time interval between the two). This corresponds to options (a) and (b) in claim 1, and results are shown below. The KFRE values are as for the previous table, again for Δt=5 years, and for Stage 3 to 5 patients.

| Feature set | Training data | Verification data |
|---|---|---|
| Core set 2 | CKD algo: AUC = 0.937 | CKD algo: 0.924 |
| | KFRE: AUC = 0.893 | KFRE: AUC = 0.896 |
| Core set 2 + | CKD algo: AUC = 0.94 | CKD algo: AUC = 0.929 |
| age | KFRE: AUC = 0.893 | KFRE: AUC = 0.896 |
| Core set 2 + | CKD algo: AUC = 0.954 | CKD algo: AUC = 0.94 |
| add'l features | KFRE: AUC = 0.893 | KFRE: AUC = 0.896 |

From this, it can be seen that the use of the presently provided predictive model gives rise to superior performance over KRFE in all settings. It will be noted that an improvement is demonstrated when the feature set comprises only the core set of features, and that a further improvement is achieved when additionally including age, and the additional features set out above. It is envisaged that similar results would be obtained when substituting creatinine measurements for eGFR values in the above, since the two are approximately proportional to each other.

As discussed, the present invention is not only highly effective for predicting a probability of kidney failure in patients with Stage 3 to 5 CKD, it is also useful for making predictions for patients having Stage 1 or 2 CKD. Results of these experiments are shown in the table below.

| Feature set | Training data | Verification data |
|---|---|---|
| Core set 1 | AUC = 0.811 | AUC = 0.760 |
| Core set 1 + age | AUC = 0.826 | AUC = 0.787 |
| Core set 1 + add'l | AUC = 0.875 | AUC = 0.859 |
| features | | |

In the table above, it will be appreciated that there are no comparative examples based on KFRE calculations, since KFRE was designed to operate on stages 3-5 only. The values shown in the table are the AUC values obtained using a gradient-boosted decision tree algorithm as described above.

### Experimental Methods & Results—Neural Network Model

Similar methods were used to assess the performance of a neural network model, rather than a gradient-boosted decision trees algorithm. Specifically, a multi-layered perceptron having 4 layers of 256 nodes, using mish activations were each trained using an Adam optimizer on a multi-target cross entropy loss. The neural network model was trained on a UK-based training data set containing data relating to approximately 850,000 patients having CKD. Data was obtained for Δt=3 years, and Δt=5 years. The data was cross-validated using the same UK-based training data set, hence the marginally better results.

A similar set of results was obtained as for the gradient-boosted decision trees.

“Creatinine” (corresponding to the recent creatinine level) and “Creatinine_Slope” were identified as a core features set “Core Set 1”, which covers options (c) and (d) of claim 1. Additional informative features are as follows, referred to collectively below as “add'l features”.
Albumin (albumin level)
Last_ACR (recent albumin-to-creatinine ratio)
CKD Diag (CKD diagnosis status)
HbA1c (glycated haemoglobin level)
Haemoglobin (haemoglobin level)
BP Systolic (systolic blood pressure)
Patient_gender (the gender of the patient)

Results are shown in the table below, for a prediction for Δt=3 years and 5 years. These results were obtained from patients with Stage 3 to 5 CKD.

| | Training data | Training data |
|---|---|---|
| Feature set | Δt = 3 years | Δt = 5 years |
| Core set 1 | CKD algo: AUC = 0.958 | CKD algo: AUC = 0.954 |
| | KFRE: AUC = 0.947 | KFRE: AUC = 0.936 |
| Core set 1 + | CKD algo: AUC = 0.960 | CKD algo: AUC = 0.956 |
| age | KFRE: AUC = 0.947 | KFRE: AUC = 0.936 |
| Core set 1 + | CKD algo: AUC = 0.967 | CKD algo: AUC = 0.965 |
| add'l features | KFRE: AUC = 0.947 | KFRE: AUC = 0.936 |

Core set 2 refers to a combination of a recent creatinine level and a historical creatinine value (and implicitly, the time interval between the two). This corresponds to options (a) and (b) in claim 1, and results are shown below. The KFRE values are as for the previous table, again for Δt=3 years and 5 years, and for Stage 3 to 5 patients.

| | Training data | Training data |
|---|---|---|
| Feature set | Δt = 3 years | Δt = 5 years |
| Core set 2 | CKD algo: AUC = 0.960 | CKD algo: AUC = 0.956 |
| | KFRE: AUC = 0.947 | KFRE: AUC = 0.936 |
| Core set 2 + | CKD algo: AUC = 0.962 | CKD algo: AUC = 0.958 |
| age | KFRE: AUC = 0.947 | KFRE: AUC = 0.936 |
| Core set 2 + | CKD algo: AUC = 0.968 | CKD algo: AUC = 0.965 |
| add'l features | KFRE: AUC = 0.947 | KFRE: AUC = 0.936 |

The results in the table below are similar, with Δt=3 years and 5 years, but were obtained for Stage 1 and 2 patients.

| | Training data | Training data |
|---|---|---|
| Feature set | Δt = 3 years | Δt = 5 years |
| Core set | 0.718 | 0.704 |
| Core set + age | 0.696 | 0.700 |
| Core set + add'l | 0.842 | 0.844 |
| features | | |
| Core set 2 | 0.793 | 0.775 |
| Core set 2 + age | 0.775 | 0.769 |
| Core set 2 + add'l | 0.859 | 0.858 |
| features | | |

In the table above, it will be appreciated that there are no comparative examples based on KFRE calculations, since KFRE was designed to operate on stages 3-5 only. The values shown in the table are the AUC values obtained using a neural network model as described above.

### GENERAL STATEMENTS ABOUT THE APPLICATION

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. A computer-implemented method of determining, at a prediction time tₚ, likelihood of kidney failure of a patient within an amount of time Δt, the computer-implemented comprising:
receiving input data, the input data comprising a recent creatinine level c_{R} or recent eGFR eGFR_{R}, and one or more of the following:
(a) an initial creatinine level c₀ and either: a time t₀ at which the initial creatinine level c₀ was measured, or a time interval ΔT₀=tₚ−t₀;
(b) an initial estimated glomerular filtration rate (eGFR) eGFR₀ and either: a time t₀ at which the initial eGFR was determined, or a time interval ΔT₀=tₚ−t₀;
(c) for a plurality of past creatinine level measurements cᵢ measured at a respective times tᵢ, a statistical parameter derived from a linear regression of the plurality of past creatinine level measurements; and
(d) for a plurality of past eGFR values eGFRᵢ determined at respective times tᵢ, a statistical parameter derived from a linear regression of the plurality of past eGFR values; and
applying a machine-learning model to the input data to generate an output indicating the likelihood of kidney failure within the given amount of time Δt.

2. A computer-implemented method according to claim 1, wherein:
the machine-learning model comprises a gradient-boosted decision trees algorithm or a neural network model.

3. A computer-implemented method according to claim 1, wherein:
the statistical parameter comprises one or more of: a slope with respect to time; an error calculated from a sum of residuals; an intercept; a number of points considered when constructing the linear regression; and a variance.

4. A computer-implemented method according to claim 1, wherein:
the input data comprises (b) and/or (d);
the or each the eGFR value eGFR₀ is calculated from a corresponding creatinine level c₀ and additional patient data comprising one or more of: age, sex, race, body size, blood urea nitrogen measurement, and serum albumin measurement.

5. A computer-implemented method according to claim 1, wherein:
the input data further comprises one or more of: age, albumin to creatinine ratio, serum albumin, serum cystatin-c, serum phosphate, serum bicarbonate, serum calcium, haemoglobin, glycated haemoglobin, blood urea nitrogen, number of acute kidney injury events, systolic blood pressure, diastolic blood pressure, resting heart rate, diabetes status, hypertension status, CKD diagnosis status; and patient's gender.

6. A computer-implemented method according to claim 5, wherein:
the input data comprises:
the recent creatinine level c_{R}; and
the patient's age; and
one or more of:
the initial creatinine level c₀ and either: a time t₀ at which the initial creatinine level c₀ was measured, or a time interval ΔT₀=tₚ−t₀; and
for a plurality of past creatinine level measurements cᵢ measured at a respective times tᵢ, a slope s of the linear regression over time.

7. A computer-implemented method according to claim 5, wherein:
the input data comprises:
the recent creatinine level c_{R};
albumin-to-creatinine ratio;
serum albumin;
haemoglobin;
glycated haemoglobin;
systolic blood pressure;
CKD diagnosis status;
patient's gender; and
one or more of:
the initial creatinine level c₀ and either: a time t₀ at which the initial creatinine level c₀ was measured, or a time interval ΔT₀=tₚ−t₀; and
for a plurality of past creatinine level measurements cᵢ measured at a respective times tᵢ, a slope s of the linear regression over time.

8. A computer-implemented method according to claim 6, wherein:
the input data further comprises blood urea nitrogen.

9. A computer-implemented method according to claim 1, wherein:
the amount of time Δt is 1 to 10 years; or
the input data further comprising the value of Δt, which is selectable by a user of the computer-implemented method.

10. A computer-implemented method according to claim 1, further comprising:
determining, based on the output of the machine-learning model, whether the patient is a fast progressor or a slow progressor.

11. A computer-implemented method according to claim 1, wherein:
either:
the patient has been diagnosed with Stage 1 or Stage 2 chronic kidney disease (CKD);
the patient has been diagnosed with Stage 3, Stage 4, or Stage 5 CKD; or
the patient has not been diagnosed with CKD.

12. A computer-implemented method of generating a machine-learning model configured to determine, at a prediction time tₚ, a likelihood of kidney failure of a patient within a given amount of time Δt, the computer-implemented method comprising:
receiving training data, the training data comprising a plurality of data sets, representing a plurality of patients, each data set comprising input data and output data, wherein for the j^{th} data set: the input data comprises a recent creatinine level c_{j,R} or a recent eGFR eGFR_{j,R} and:
(a) a historical creatinine level c_{j,H}, and the time t_{j,H} at which it was obtained;
(b) a historical eGFR eGFR_{j,H}, and the time t_{j,H} at which it was obtained;
(c) for a plurality of past creatinine levels measured at respective times tᵢⱼ, a statistical parameter derived from a linear regression of the plurality of past creatinine level measurements; and
(d) for a plurality of past eGFR values eGFRᵢ determined at respective times tᵢ, a statistical parameter derived from a linear regression of the plurality of past eGFR values; the output data comprises an indication of an interval Δtⱼ between the time tⱼ of kidney failure, and the time of measurement of c_{j,R}; and
training the machine-learning model using the training data.

13. A computer-implemented method according to claim 10, wherein:
the plurality of data sets may include one or more clusters of data sets, wherein each cluster comprises a plurality of input data items and a respective plurality of corresponding output data items, the input data items and output data items in each cluster corresponding to data obtained at different times or over different timescales for the same patient.

14. A computer-implemented method according to claim 11, wherein:
the patients for whom there is an associated cluster of data sets include patients diagnosed with end-stage renal disorder (ESRD).

15. A computer-implemented method according to claim 10, wherein:
the training data comprises a further plurality of pairs of data, wherein for the k^{th} further pair:
the input data comprises a recent creatinine level c_{k,R} and one or more of:
(e) a historical creatinine level c_{k,H}, and the time t_{k,H} at which it was obtained;
(f) a historical eGFR eGFR_{k,H}, and the time t_{k,H} at which it was obtained;
(g) for a plurality of past creatinine levels cₖᵢ measured at respective times tₖᵢ, a statistical parameter determined from a linear regression of the plurality of past creatinine level measurements; and
(h) for a plurality of past eGFR values eGFRₖᵢ determined at respective times tₖᵢ, a statistical parameter derived from a linear regression of the plurality of past eGFR values;
the output data comprises an indication that kidney failure has not occurred within an interval of Δtₖ since the time of measurement of c_{k,R}.

16. A computer-implemented method according to claim 1, wherein:
the machine-learning model is generated using the computer-implemented method of claim 12.

17. A kidney failure likelihood determination system configured to determine, at a prediction time tₚ, a likelihood of kidney failure of a patient within an amount of time Δt, the system comprising a processor which is configured to perform the method of claim 1.
